(19) United States
(12) Patent Application Publication
(10) Pub. No.: US 2003/0022150 A1
(43) Pub. Date: Jan. 30, 2003

(54) Methods for detecting a target molecule

(76) Inventors: Jeffrey R. Sampson, Burlingame, CA (US); Gary B. Gordon, Saratoga, CA (US); Kevin J. Luebke, Dallas, TX (US); Joel Myerson, Berkeley, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 09/915,044

(22) Filed: Jul. 24, 2001

(51) Int. Cl. C12Q 1/00; C12Q 1/68; G06F 19/00; G01N 33/48; G01N 33/50; G01N 27/26

(52) U.S. Cl. 435/4; 435/6; 205/777.5; 702/19

(57) ABSTRACT

A method for detecting a target moiety is disclosed. In one embodiment, a plurality of electrodes supported by a semiconductor substrate are brought into proximity with a reaction medium comprising a sample suspected of containing the target molecule. Each of the electrodes comprises at least one target probe. A plurality of cells within the semiconductor substrate are selectively addressed to apply a stimulus to each of the electrodes to activate a predetermined redox active moiety that is associated with an electrode and to detect, by means of the electrodes, corresponding responses produced as a result of the activation of the redox active moieties. The magnitude of the corresponding responses indicates the presence or absence of the target molecule in the sample.

### BACKGROUND OF THE INVENTION

### FIELD OF THE INVENTION

This invention relates to the field of bioscience in which arrays of oligonucleotide probes, fabricated or deposited on a surface, are used to identify DNA sequences in cell matter. The present invention has a wide range of application for the use of arrays for conducting cell study, for diagnosing disease, identifying gene expression, monitoring drug response, determination of viral load, identifying genetic polymorphisms, and the like. Within the drug discovery process there exists a growing need for accurate, automated and high throughput systems for performing genetic mutation analysis. As enabling technologies are developed and become accessible, it is likely that such analyses will migrate into the clinical diagnostic markets. A successful entry into this setting will require that the necessary methods and instrumentation be accurate, cost effective and most of all, easy-to-use.

Significant morbidity and mortality are associated with infectious diseases and genetically inherited disorders. More rapid and accurate diagnostic methods are required for better monitoring and treatment of these conditions. Molecular methods using DNA probes, nucleic acid hybridization and in vitro amplification techniques are promising methods offering advantages to conventional methods used for patient diagnoses.

Nucleic acid hybridization has been employed for investigating the identity and establishing the presence of nucleic acids. Hybridization is based on complementary base pairing. When complementary single stranded nucleic acids are incubated together, the complementary base sequences pair to form double-stranded hybrid molecules. The ability of single stranded deoxyribonucleic acid (ssDNA) or ribonucleic acid (RNA) to form a hydrogen bonded structure with a complementary nucleic acid sequence has been employed as an analytical tool in molecular biology research. The availability of radioactive nucleoside triphosphates of high specific activity and the development of methods for their incorporation into DNA and RNA has made it possible to identify, isolate, and characterize various nucleic acid sequences of biological interest. Nucleic acid hybridization has great potential in diagnosing disease states associated with unique nucleic acid sequences. These unique nucleic acid sequences may result from genetic or environmental change in DNA by insertions, deletions, point mutations, or by acquiring foreign DNA or RNA by means of infection by bacteria, molds, fungi, and viruses.

The application of nucleic acid hybridization as a diagnostic tool in clinical medicine is limited due to the cost and effort associated with the development of sufficiently sensitive and specific methods for detecting potentially low concentrations of disease-related DNA or RNA present in the complex mixture of nucleic acid sequences found in patient samples.

One method for detecting specific nucleic acid sequences generally involves immobilization of the target nucleic acid on a solid support such as nitrocellulose paper, cellulose paper, diazotized paper, or a nylon membrane. After the target nucleic acid is fixed on the support, the support is contacted with a suitably labeled probe nucleic acid for about two to forty-eight hours. After the above time period, the solid support is washed several times at a controlled temperature to remove unhybridized probe. The support is then dried and the hybridized material is detected by autoradiography or by spectrometric methods. When very low concentrations must be detected, the above method is slow and labor intensive, and nonisotopic labels that are less readily detected than radiolabels are frequently not suitable. The above time period may be shortened by employing techniques such as electrophoresis, which allows detection of specific nucleic acid sequences in a relatively shorter time of about 10 minutes to one hour.

A method for the enzymatic amplification of specific segments of DNA known as the polymerase chain reaction (PCR) method has been described. This in vitro amplification procedure is based on repeated cycles of denaturation, oligonucleotide primer annealing, and primer extension by thermophilic polymerase, resulting in the exponential increase in copies of the region flanked by the primers. The PCR primers, which anneal to opposite strands of the DNA, are positioned so that the polymerase catalyzed extension product of one primer can serve as a template strand for the other, leading to the accumulation of a discrete fragment whose length is defined by the distance between the 5′ ends of the oligonucleotide primers.

Other methods for amplifying nucleic acids have also been developed. These methods include single primer amplification, ligase chain reaction (LCR), transcription-mediated amplification methods including 3SR and NASBA, the Q-beta-replicase method, the rolling circle amplification, and so forth. Regardless of the amplification used, the amplified product must be detected.

One method for detecting nucleic acids is to employ nucleic acid probes that have sequences complementary to sequences in the target nucleic acid. A nucleic acid probe may be, or may be capable of being, labeled with a reporter group or may be, or may be capable of becoming, bound to a support. Detection of signal depends upon the nature of the label or reporter group. Usually, the probe is comprised of natural nucleotides such as ribonucleotides and deoxyribonucleotides and their derivatives although unnatural nucleotide mimetics such as 2′-modified nucleosides, peptide nucleic acids and oligomeric nucleoside phosphonates are also used. Commonly, binding of the probes to the target is detected by means of a label incorporated into the probe. Alternatively, the probe may be unlabeled and the target nucleic acid labeled. Binding can be detected by separating the bound probe or target from the free probe or target and detecting the label. In one approach, a sandwich is formed comprised of one probe, which may be labeled, the target and a probe that is or can become bound to a surface. Alternatively, binding can be detected by a change in the signal-producing properties of the label upon binding, such as a change in the emission efficiency of a fluorescent or chemiluminescent label. This permits detection to be carried out without a separation step. Finally, binding can be detected by labeling the target, allowing the target to hybridize to a surface-bound probe, washing away the unbound target and detecting the labeled target that remains.

Direct detection of labeled target hybridized to surface-bound probes is particularly advantageous if the surface contains a mosaic of different probes that are individually localized to discrete, known areas of the surface. Such ordered arrays containing a large number of oligonucleotide probes have been developed as tools for high throughput analyses of genotype and gene expression. Oligonucleotides synthesized on a solid support recognize uniquely complementary nucleic acids by hybridization, and arrays can be designed to define specific target sequences, analyze gene expression patterns or identify specific allelic variations.

In one approach, cell matter is lysed, to release its DNA as fragments, which are then separated out by electrophoresis or other means, and then tagged with a fluorescent or other label. The resulting DNA mix is exposed to an array of oligonucleotide probes, whereupon selective attachment to matching probe sites takes place. The array is then washed and imaged so as to reveal for analysis and interpretation the sites where attachment occurred.

Desirably, the array is a matrix of the order of 10,000 probe sites or more in an area several to tens of millimeters on a side. Each oligonucleotide probe has a length typically in the 10 to 40 base pair length.

DNA array technology provides an effective platform for detecting and interrogating nucleic acid sequences. Of particular interest are DNA arrays consisting of short DNA oligonucleotides or longer cDNAs attached to a surface in a spatially addressed manner. A target nucleic acid sequence can then be interrogated making use of the inherent property of nucleic acids to form hydrogen-bonded duplexes according Watson and Crick base-pairing rules. Although arrays have the benefit of being highly sensitive and accurate when developed for specific target sequences, their implementation generally relies on the use of relatively long DNA probes (>15 mers) in order to form stable, and therefore detectable target/probe duplexes. Unfortunately, this length of probe restricts the utility of any given array since it is not possible to physically display a sequence-complete set of this length.

One potential solution to the feature density problem is to use short oligonucleotide probes in conjunction with enzymatic processes. For example, It is known in the art that some DNA polymerases can extend short (>6-mer) oligonucleotide primers when hybridized to longer (>12-mer) targets. (See U.S. Pat. Nos. 5,595,890 and 5,534,424). Likewise, some DNA ligases can ligate together duplex composed of short (>6-mer) oligonucleotides as revealed in U.S. Pat. Nos. 4,988,617 and 5,494,810.

Array systems generally rely on optical methods for detecting the hybridized target. Although optical detection can be very sensitive, it generally requires sophisticated instrumentation. For example, fluorescence methods can detect sub-femtomole quantities of fluorescent dye. However they usually require expensive lasers for dye excitation and a complex light collecting and focusing system in order to reach high levels of sensitivity. Moreover, optical instruments usually scan the array one feature at a time. Thus, even though the scan rate can be quite fast (msec./feature), the total array scan time required for higher density arrays will be significant. Finally because most hybridization array-based systems detect dye moieties within the target, the dyes need to be incorporated into the target molecule prior to hybridization. This additional level of complexity in the system can limit its utility.

A system is desired which obviates the problems associated with probe length and optical detection methods.

### SUMMARY OF THE INVENTION

One embodiment of the present invention is a method for detecting a target molecule. A plurality of electrodes supported by a semiconductor substrate are brought into proximity with a reaction medium comprising a sample suspected of containing the target molecule. Each of the electrodes comprises at least one target probe. A plurality of cells within the semiconductor substrate are selectively addressed to apply a stimulus to each of the electrodes to activate a predetermined redox active moiety that is associated with an electrode and to detect, by means of the electrodes, corresponding responses produced as a result of the activation of the redox active moieties. The magnitude of the corresponding responses indicates the presence or absence of the target molecule in the sample.

Another embodiment of the present invention is a method for detecting a target molecule. A plurality of electrodes supported by a semiconductor substrate is brought into proximity with a reaction medium comprising a sample suspected of containing the target molecule. Each of the electrodes comprises at least one target probe. Electrical signals are selectively applied to each of the electrodes to activate a redox active moiety that is associated with each of the target probes. Corresponding electrical signals produced as a result of the activation of the redox active moieties are detected by means of the electrodes. The magnitude of the corresponding electrical signals indicates the presence or absence of the target molecule.

Another embodiment of the present invention is a method for detecting a target nucleic acid. A plurality of electrodes supported by a semiconductor substrate is brought into proximity with a reaction medium comprising a sample suspected of containing the target molecule. Each of the electrodes comprises at least one oligonucleotide probe. An item of numerical data is sent to each of a plurality of cells within the semiconductor substrate by means of a data bus. The item of numerical data participates in the selection of a voltage to be applied to the electrodes. An address is sent to address decoders, which are on said semiconductor substrate and are in communication with the plurality of cells. In this way, electrical signals are selectively applied to each of the electrodes to activate a redox active moiety that is associated with each of the oligonucleotide probes. Corresponding electrical signals produced as a result of the activation of the redox active moieties are detected by means of the electrodes. The magnitude of the corresponding electrical signals indicates the presence or absence of the target nucleic acid.

Another embodiment of the present invention is a method for detecting a target nucleic acid. A plurality of electrodes supported by a semiconductor substrate is brought into proximity with a reaction medium comprising a sample suspected of containing the target molecule. Each of the electrodes comprises at least one oligonucleotide probe wherein each of the oligonucleotide probes comprises a redox active moiety. An item of numerical data is sent to storage means in each of a plurality of cells within the semiconductor substrate by means of a data bus. The item of numerical data participates in the selection of a voltage to be applied to the electrodes. An address is sent to address decoders, which are on said semiconductor substrate and are in communication with said storage means. In this way, the item of numerical data is stored in the storage means and voltages are selectively applied to each of the electrodes to activate the redox active moieties. Corresponding current or difference in potential or a combination thereof produced as a result of the activation of the redox active moieties are detected by means of the electrodes. The magnitude of the current or potential difference indicates the presence or absence of the target nucleic acid.

Another embodiment of the present invention is a method for detecting a target nucleic acid. A plurality of electrodes supported by a semiconductor substrate is brought into proximity with a reaction medium comprising a sample suspected of containing the target molecule. Each of the electrodes comprises at least one oligonucleotide probe. A polymerase extension reaction is conducted to incorporate a redox active moiety or precursor thereof in a target dependent manner, i.e., to incorporate a redox active moiety or precursor thereof in each of the oligonucleotide probes to which a target nucleic acid is bound. An item of numerical data is sent to storage means in each of a plurality of cells within the semiconductor substrate by means of a data bus. The item of numerical data participates in the selection of a voltage to be applied to the electrodes. An address is sent to address decoders, which are on said semiconductor substrate and are in communication with said storage means. In this way, the item of numerical data is stored in the storage means and voltages are selectively applied to each of the electrodes to activate the redox active moieties. Corresponding current or difference in potential or a combination thereof produced as a result of the activation of the redox active moieties is detected by means of the electrodes. The magnitude of the current or potential difference indicates the presence or absence of the target nucleic acid.

Another embodiment of the present invention is a method for identifying target nucleic acids in a sample. The sample is applied to a plurality of test sites. Each of the test sites comprises an oligonucleotide probe attached to an electrode. Each of the electrodes is part of a surface of an integrated circuit. Each of the oligonucleotide probes is capable of specifically binding to a target nucleic acid molecular structure, such that each of the test sites has oligonucleotide probes which specifically bind to a different target molecular structure. The sample is incubated on the test sites in the presence of a polymerase and nucleotide triphosphate analogs comprising a redox active moiety or a precursor thereof to extend each oligonucleotide probe and to associate the redox active moiety or precursor thereof with each extended oligonucleotide probe. A proviso is that, when the nucleotide triphosphate analogs comprise a precursor, a binding partner for the precursor is added wherein the binding partner comprises a redox active moiety. A voltage is applied to each of the test sites by means of circuitry associated with the integrated circuit wherein the voltage is sufficient to activate the redox active moiety. The next step involves detecting by means of the integrated circuit a current or difference in potential or a combination thereof at each of the test sites, the magnitude of which is related to the presence of the target nucleic acids.

In the above embodiment the integrated circuit may comprise a semiconductor substrate supporting a plurality of electrodes and a plurality of cells within the semiconductor substrate. The integrated circuit may comprise a substrate that includes a plurality of digital analog converters, each electrically coupled to a respective electrode and each being associated with a respective cell, address decoders in communication with each of the cells, a data bus for delivering binary numerical data to each of the cells, address buses for delivering addresses to the address decoders, and storage means in each of the cells for storing the numerical data. The storage means is in communication with the digital analog converter in the cell. The steps may further comprise sending binary numerical data to the storage means of each the cells by means of the data bus, the binary numerical data being representative of an electrical signal, sending addresses to the address decoders whereby the binary numerical data is stored in the storage means and electric signals are selectively applied to each of the electrodes by means of the digital analog converters to activate the redox active moiety and detecting, by means of the electrodes, corresponding electrical signals produced as a result of the activation of the redox active moieties, the magnitude of the corresponding electrical signals indicating the presence or absence of the target molecule.

Another embodiment of the present invention is a method of testing a sample for the presence of target nucleic acids. A sample is applied to an array of test sites in multiple locations on a surface of an integrated circuit, each site having oligonucleotide probes formed therein of known binding characteristics. The oligonucleotide probes in each test site differ from the oligonucleotide probes in other test sites in a known predetermined manner such that the test site location of oligonucleotide probes and their binding characteristics are known. Each test site is treated to extend the length of each oligonucleotide probe thereby incorporating an electronically responsive detector agent into each of the oligonucleotides. An electrical signal is applied to each of the test sites by means of circuitry associated with the integrated circuit. The electrical signal is sufficient to activate the electronically responsive detector agent. A change in electronic properties of the test sites resulting from the binding of target nucleic acid to lengthened oligonucleotide probes in the test sites is detected by detection circuitry coupled to individual test sites to determine which target nucleic acid has bound to a test site. The presence of a multiplicity of different target nucleic acids in the sample is detected.

Another embodiment of the present invention is a device comprising (a) a semiconductor substrate, (b) at least one surface having associated therewith a redox active moiety, (c) an electrode adjacent the surface and supported by the semiconductor substrate, (d) a cell within the semiconductor substrate, (e) a digital-to-analog converter to which the electrode is electrically coupled, the digital analog converter being associated with the cell, (f) an address decoder in communication with the cell, (g) a data bus for delivering an item of numerical data to the cell, (h) an address bus for delivering an address to the address decoder, and (i) means for monitoring the surface.

### BRIEF DESCRIPTION OF THE DRAWINGS

FIG. 1 is a schematic diagram depicting a device in accordance with the present invention.

FIG. 2 is a schematic diagram depicting the cell of the device of FIG. 1.

FIG. 3 is a schematic diagram depicting an aspect of a device in accordance with the present invention; and

FIG. 4 is a flow chart depiction of an embodiment of the present invention.

### DETAILED DESCRIPTION OF THE INVENTION

The present invention relates to methods and devices for detecting a target molecule and, more particularly, to the electronic detection of such a target molecule. The method utilizes a device that may be prepared by attaching a target probe, e.g. a short oligonucleotide, to an array of electrodes, each of which is part of an integrated circuit (IC) which activates and monitors the electrochemical activity at the electrode surface. The IC architecture is such that a large number of electrodes can be specifically addressed through a relatively small number of externally accessible leads on an array chip. The sample, e.g. a target nucleic acid, is applied to the probe array surface whereby the target molecule either modulates the electronic properties of a detector agent associated with the probes or mediates an enzymatic process, which incorporates a detector agent into the probes. The electrochemical properties of the probes at each electrode are then monitored using the drive and sense circuitry associated with each probe electrode on the chip array.

A stimulus-response system is employed for each electrode of the array of electrodes. The stimulus may be voltage or current and the response may be current or voltage, respectively. There are numerous ways in which the stimulus may be applied and the response measured as discussed in more detail below. Accordingly, voltammetry, amperometry, potentiometry, and so forth may be performed on each electrode to detect the result of a target binding event.

In the present devices there are electronics underlying each electrode that are in communication with addressing sites and measuring sites that may be on or off the array of electrodes itself. Appropriate wires are employed to address each site individually so that the application of a stimulus to each site may be accomplished in a predetermined manner. Similarly, wires are employed to receive a response from each site in a predetermined manner. A computer may be used to control the addressing of the sites and to record the results.

The devices and methods of this invention allow important diagnostic reactions to be carried out under complete electronic control. The basic concept of this invention is a micro-electronic device with specifically designed addressable microscopic locations. Each micro-location has a derivatized surface for the attachment of specific binding entities. After the initial fabrication of the basic microelectronic structure, the device is able to self-direct the addressing of each specific micro-location with specific binding entities.

In the present invention, the electrochemical forces are controlled by an array of electrodes and driven by an integral integrated circuit which uses random access memory technology (RAM).

The following discussion is by way of illustration and not limitation.

Devices for Carrying Out the Methods of the Invention

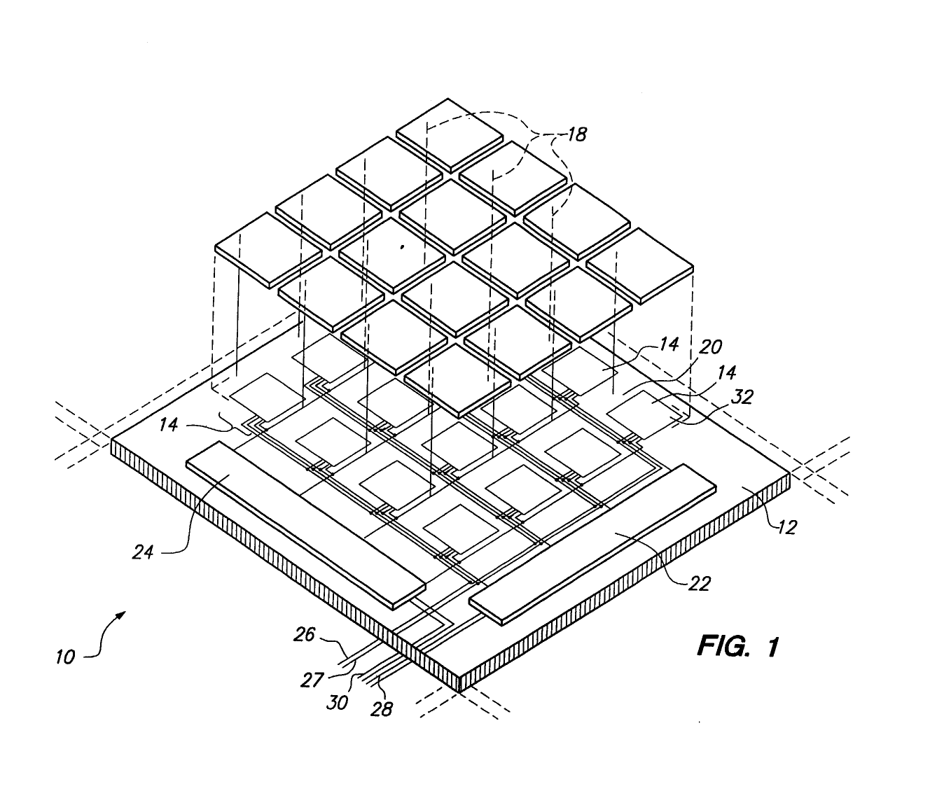

Referring to FIG. 1, a device 10 is depicted. A semiconductor substrate 12 has a plurality of cells 14 such as RAM cells within the semiconductor substrate 12. A plurality of digital analog converters (not shown) is each associated respectively with a cell 14. Each of the digital analog converters (not shown) is respectively electrically coupled to an electrode or microelectrode 18, which is supported by semiconductor substrate 12. The electrode 18 is destined to have affixed thereto a receptor or target probe 58 [FIG. 3]. The electrical coupling is achieved by means of, for example, conventional inter-layer metallic “vias.” FIG. 1 depicts optional buffer amplifier 20, which functions to isolate the digital analog converters (not shown) of cells 14 from electrical loads applied to their electrodes 18. Address decoders 22 and 24 are in communication with each of the cells 14 by means of, for example, conductive metallization interconnection paths. Data bus 26 and 27 is in communication with each of cells 14 by similar means. The data bus 26 and 27 delivers numerical data to each of cells 14. Also included are address buses 28 and 30, which deliver addresses to address decoders 22 and 24, respectively, and are in communication therewith by means similar to that described above for the address decoders and the data bus.

In a preferred embodiment, data such as numerical data is sent to each of cells 14 by means of the data bus. The numerical data is representative of an electric signal as explained in more detail below. Addresses are sent to address decoders 22 and 24 whereby electric signals are selectively applied to each of the electrodes 18. The numerical data may be, for example, binary numerical data; in a simple case representative of values 0 volts and 3 volts, and so forth. In the latter case the data are represented by a single bit, 0 or 1 (“0”→0 volts and “1”→3 volts).

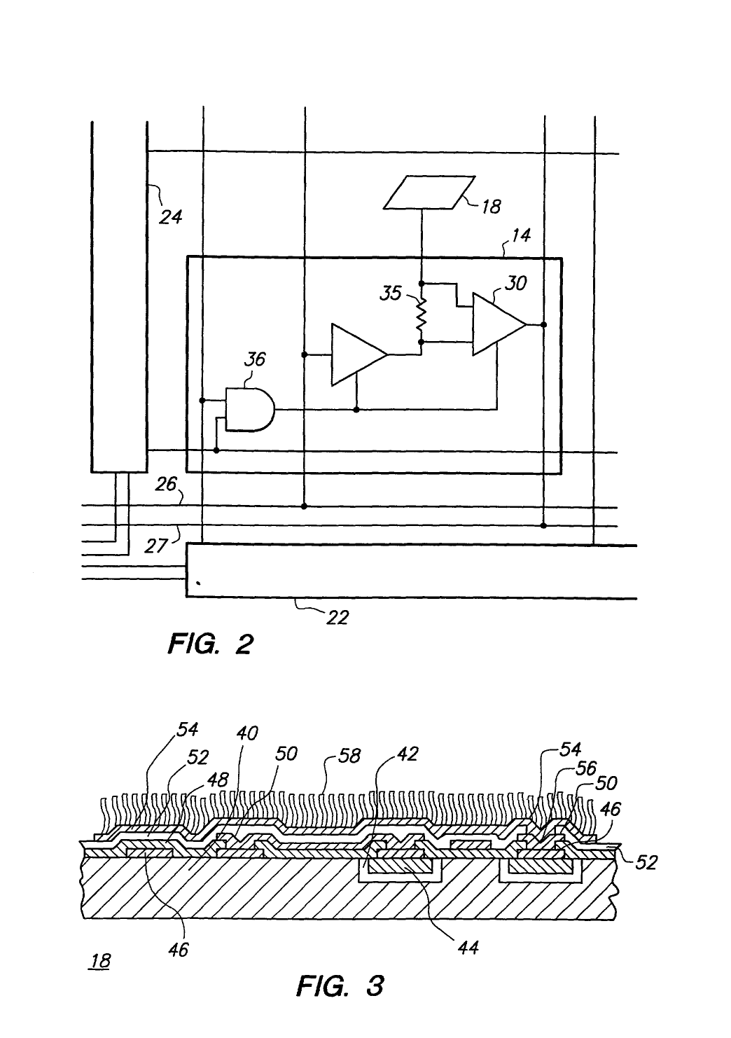

Referring to FIG. 2, a single cell 14 is interrogated at a time. The cell is addressed as already described by the coincidence of signals from the x and y address decoders 22, 24. Once addressed, the cell 14 is connected to the drive and sense lines 26, 27 communicating with the chip 12. Drive signals applied to drive line 26 may be cyclic signals typical of those used in voltammetry. The addressed cell 14 applies that drive to its electrode 18. The resultant current flowing is sensed by differential amplifier 30 as the voltage drop produced across series-resistor 35. The measured current is gated onto the sense bus 27 by amplifier 30 when its enable line coming from coincidence-gate 36 goes to a logical “1” state. As a consequence, conventional voltammetry measurements, for example, of any site may be made using the bus signals 26, 27 that communicate with the chip 12.

The purpose of the present invention is to accomplish a volt-amperometric or similar measurement at any or all of thousands of sites, without the overhead of thousands of interconnects.

Variations to the present invention include:

(1) semi-parallel configurations, for reading several cells at a time, to speed up the assay. In one embodiment, with off-chip reading, ten sense lines can exit the chip 12, each servicing 10% of the available sites; with a shortening of the time required to read the chip 12 by a factor of ten;

(2) moving all or part of the cyclic voltammetry circuitry onto the chip 12 such as the drive-waveform generator;

(3) making a determination on the chip 12 as to the significance of the voltammetry reading, such as the degree of binding it indicates; and sending that interpreted information off of the chip 12, this determination can be made at each site, or alternatively at central locations servicing a number of sites; one way of making the determination would be to measure the current drawn by the electrode at two voltages, and then reporting off-chip the difference between the two voltages; (this reduces the off-chip reporting for one site to a single number indicative of the binding; rather than the complete V-I curve; a alternatively, the chip could fit a line or polynomial curve to the V-I curve, and merely report off-chip the line or curve's parameters; the parameters for a line could be slope and offset; or two end-points; the parameters for a curve are its polynomial coefficients).

The array chip 12 may contain a relatively small number of microelectrodes 18 in an array, e.g. less than 50, or may contain an array of a large number of microelectrodes 18, e.g. several thousand to several hundred thousand. The array may also contain a small Pt plate and micro Ag/AgCl (saturated with KCl) electrode to serve as the counter and reference electrodes, respectively. Alternative micro-counter and micro-reference electrodes known in the art may also be included. The electronic control of the individual microelectrodes 18 is provided so as to control the voltage or current. When one aspect is set, the other may be monitored. For example, when the voltage is set, the current may be monitored. The voltage and/or current may be applied in a direct current mode or may vary with time.

The devices used in the present invention may be fabricated according to procedures well-known to those skilled in the art of digital and IC design. Reference books that are exemplary of those directed to the above include VLSI Technology by S.M. Sze (1988) ISBN 0-07-062735-5 and Basic VLSI Design by Pucknell and Eshraghian (1988) ISBN 0 7248 0105 7. Typical integrated circuits use two to five or more layers of interconnection metal with insulator layers in between. Modern IC's usually use an aluminum alloy for metallization in conjunction with “vias” of a tungsten alloy. The metal layers are generally of a thickness on the order of approximately about 0.1 to about 1 micron.

FIG. 3 depicts, in cross-section, a portion of a device in accordance with the present invention showing an electrode assembly 18. P-substrate 40 contains depletion regions 42 and N-diffusion regions 44. Metal layer 46 is formed from a selected metal and is found within insulator layer 48. Above 46 lies metal layer 50, which is found within insulator layer 52. The upper most layer 54 is also formed from a selected metal and is the outer layer of the electrode 18. Affixed to layer 54 is a receptor or target probe 58, e.g. a DNA or RNA receptor probe. The target probe 58 at each microelectrode must be capable of binding to a known molecular or cellular target, e.g. DNA. Typically, each probe 58 at a given microelectrode binds to a different target, or different target molecular structure according to the types of target desired to be detected. Oligonucleotides, single or double stranded DNA or RNA, antibody or peptide test probes known to those skilled in the art may be used.

Via 56 is formed by the interconnection of 46, 50, and 54, which may be referred to as metallization layers. In some systems, it may be fabricated so as to lie between the electrodes and not underlying them as shown in FIG. 3.

Gold may be employed for one or more metallization layers. For cost reasons in the present invention aluminum is preferred for the intermediate metal layers and gold for the top layer, which would be the electrodes. Fixed electrodes may be plated over the processes well-known in the art of IC, with a variety of metals, including gold and nickel, chosen to be the most compatible with the oligomer primer attachment chemistry for probe 58. In addition to aluminum, suitable metals for circuitry include gold, tin, platinum, palladium, and various metal combinations.

The insulator layers are usually of thickness similar to that mentioned above for the metallization layers and are made of an insulating material, i.e., a non-conductive material such as silicon dioxide and the like. The insulator layers are grown above and intrinsically adhered to the metal layers. The overcoat layer is conveniently applied by deposition techniques, e.g., plasma enhanced chemical vapor deposition, and the like.

The connections between the electrodes and the circuit cells are provided by the interconnected layers of metal and insulator by means of holes in the insulator. See, for example, the depiction of via 46 in FIG. 3. These holes are typically on the order of fractions of microns, usually about 0.2 to about 2 microns in diameter and may be formed by microlithographic or other techniques well-known in the art of IC design such as electron beam lithography, ion beam lithography, or molecular beam epitaxy. While microscopic locations sites are desirable for some applications such as high density arrays, larger addressable sites (e.g. larger than 2 mm) may be employed.

The resultant device 10 can then be plugged into a microprocessor controlled power supply and multimeter apparatus, which controls and operates the device upon destined sample analysis. In its destined use for detection of a target sample, e.g. nucleic acid polymer, when a probe 58 (FIG. 3) bonds or hybridizes to a target nucleic acid, the dielelectric properties of the probe 58 changes and measured electrical properties of the microelectrode 18 changes in a measurable fashion via the connective circuitry to the chip 10.

The control electronics to the microelectrodes 18 can be attained in many conventional ways. For example, an adaptation of the system described in U.S. Pat. No. 5,849,486 may be used, the relevant portions of which are incorporated herein by reference. The system described in U.S. patent application Ser. No. 09/100,152, filed on Jun. 18, 1998, incorporated by reference hereunto in its entirety, is preferred. In this regard, referring to FIG. 1, the electrodes 18 of the array are at predetermined locations or sites on the integrated circuit chip 12 and generally are of the micro scale. The usual function of the electrode 18 is to apply a DC signal. By functioning to apply a DC signal is meant that an electrode is biased either positively or negatively, operating in a direct current mode. It should be noted that other types of signal application may be used. For example, the signal may be an AC signal. The AC signal may be applied at selected sites by generation of AC signal at the selected sites. On the other hand, one or more tree-like signal buses that are accessible to each and every site may be used. To each bus may be applied either or both an AC or a DC signal. Each addressed site may, as desired, connect any signal bus to its electrode in response to appropriate settings of its storage element.

The operation of the chip 12 is best understood by considering the case where the chips are processed individually. Referring to FIG. 1, less than 20 electrical connections to the chip 12 are required for a 16,384-element embodiment. Fourteen lines are required for address, 2 for data, and a few more for power and ground. The 14 address lines carry logic signals representing 2614 or 16,384 states. Since the array is most advantageously made square, 7 lines are dedicated to encoding the x-address and 7 lines are dedicated to encoding the y-address. These 7 lines are fed each to the x-address decoder and the y-address decoder.

The 7 address lines connected to each decoder can represent 2ˆ 7 or 128 states. The output of each decoder is 128 lines. Only one output line is active at a time, namely, the one representing the state of the 7 address lines. For example, a 14-bit address sent to the chip with value of 00000000000010 has a decimal value of 2. Splitting the address into two seven-bit bytes, an address of 0000010 would be sent to the x-axis decoder and an address of 0000000 would be sent to the y-axis decoder. The 0000000 sent to the y-axis decoder causes the first or lowest of its 128 output lines to become active. Accordingly, the line might be sent “high”, while the remaining 127 liens would be set “low”, which means that the line is set to a voltage of zero. The 0000010 address, binary “2”, is the third ascending state that can be represented and, thus, causes the third line of the x-axis decoder to be set active (“or high”). In this way a positive ion is attracted to the electrode governed by this cell.

FIG. 1 may be visualized as representing the lower-leftmost 16-element corner of the 16,384-element array. The nearest element has address 00000000000000; the rightmost, 00000000000011; the leftmost, 00000110000000; and the uppermost, 00000110000011.

The 128 decoded address lines from each decoder form a grid on the chip. At each intersection is a cell of circuitry and an electrode. Each cell is only addressed when both its x and y decoded address lines are active. Thus, for any applied 14-bit address, only one cell is addressed at a time.

In this embodiment, two data lines enter the chip. They are capable of representing 2ˆ 2 or 4 logic states. These will ultimately produce one of four possible voltages on whichever electrode 18 in the array happens to be addressed. More specifically, when a circuit cell is addressed, it latches the data from the data lines by means of two D-type flip-flops. This data is held, or latched, while the process proceeds to latch independent data into each of the other 16,383 cells. The state latched into each circuit cell may have a value of 00, 01, or 11. The latching is static, as opposed to dynamic, for simplicity. The operation is reminiscent of the behavior of computer random-access-memory “RAM” chips. The preferred mode of operation is as a static RAM, which means that data does not need to be periodically refreshable by read/write cycles. However, this is not a requirement. The byte length is two bits.

The state latched into each circuit cell is delivered to a digital-to-analog converter (DAC) for conversion to an analog voltage (for example, 0.1, 2, or 3 volts). This output is shown buffered by a unity-gain amplifier. However, drive requirements for electrode are so small that the amplifier may be incorporated as a functional part of the DAC itself and, in that sense, eliminated.

A beneficial feature not shown in FIG. 1 is means to electrically test the device. This is easily accomplished by adding an additional line exiting the chip 12, which is connected in parallel to every array circuit cell 14. Each cell 14 has an analog switch, which allows sequential connecting of its analog output voltages to the bus when the cell is addressed. The test and verification cycle is as follows: Each cell is written to four times, once with each of its four allowable 00, 01, 10 and 11 states. After each write, the analog bus is monitored for presence of the correct voltage.

The size of the array may be varied depending on the application as discussed above. Fewer or more elements may be employed, depending on cost considerations, the size of the sample available for analysis and the size of the electrodes necessary to obtain the required sensitivity.

Greater or fewer voltage states may be provided for on each electrode 18 as well as voltages of both polarities. In this regard the voltages may be from any value between the two positive and negative extremes of supply voltages available to the chip 12. The particular voltages selected will depend on the application in which the device 10 is used. The voltage range does not need to be represented in equal steps; for example, four binary states could be assigned values of 0, 0.5, 4.5, and 5.0 volts.

In its simplest form only two voltage levels are provided. In this approach most of the complexity of the cell vanishes and a one-bit latch is all that is required. In this form, the density of an array can be increased considerably.

In addition to the above-mentioned features, the device may also comprise identification codes, which may be either visual or electronic, to provide for interrogation of features of the device.

In one embodiment of the present invention, a plurality of analog buses is employed and the item of numerical data identifies which analog bus connects to the electrode 18. In this embodiment each site may be switched to one of the plurality of analog buses. For efficiency of operation it is desirable to be able to set sites to one of perhaps two voltages, i.e., to either attract or repel chemical species. This may be accomplished in the present invention by traversing the chip with several analog buses such as, for example, two analog buses, namely, Bus A and Bus B. Bus A might be set to 1V and Bus B might be set to 3 V and the grid set to 2 V. The grid voltage may be set by employing an on-chip DAC or by an external lead. For the sake of illustration the setting at 1 V repels a chemical species while the setting at 3 V attracts chemical species and the setting at 2 V does neither. A digital value is written to each cell of 0, 1 or 2 (in binary 00, 01, 10) indicative of whether to connect the cell's electrode by means of a switch to Bus A, Bus B or to the grid. More specifically, if the data “00” is sent to a cell, then its switch is set to connect that cell electrode to Bus A, to which we are holding 1 V, by an on-chip or especially off-chip source. On the other hand, if the data “01” is sent to a cell, then its switch is set to connect its electrode to the grid. This grid is in essence a third bus since it is proximal to all cells. The difference is that it is an electrode that is not insulated but is in proximity to or in contact with the medium. The above approach requires neither the complexity of the analog non-volatile storage mechanism nor the need to periodically refresh the cell. It should be noted that one or more of the analog buses could optionally be the system ground or other supply voltage since these are merely voltages bused to each and every cell.

Where it is desirable to store data on the present device, storage means may be employed. In such an embodiment, each of cells of the present device may comprise storage means for storing numerical data. The storage means is in communication with each cell by means similar to that mentioned above. The storage means may be similar to that known in the art such as, for example, D-type static flip-flops, a latch, a capacitor storing an analog value, and the like. The storage means may be a dynamic RAM replicator latch with a capacitor, which can store data but needs to be refreshed. The storage means may store a value representative of a voltage or merely the fact a cell was selected and the electrode is more or less switched to an analog bus. Both situations are exemplified by D-type flip-flops in conjunction with a digital bus.

Referring to FIG. 3, for direct attachment of probes 58 to the electrodes 18, the electrode surfaces 54 must be fabricated with materials capable of binding probes in a defined orientation through covalent attachment or non-covalent conjugation methods. Materials which can be incorporated into the surface 54 of the electrodes 18 to provide for direct attachment of probes 58 include electrometal materials such as gold, niobium oxide, iridium oxide, platinum, titanium, tantalum, tungsten, indium-tin oxide and other metals. These electrometals are capable of forming stable attachments directly on the plate surface through covalent linkages with organic thiol groups incorporated into the probe as described in Whitesides et al., (1990) LANGMIUR 6; 87-96 and Hickman et al., (1991), J. AM. CHEM. SOC., 113; 1128-1132, both of which are incorporated by reference herein. As an example, a synthetic oligonucleotide probe labeled with a thiol group either at the 5′ or 3′ terminus will form a stable bond with a metal such as gold, in the plate surface 54 to create an array of attached probes 58.

The oligonucleotide probes 58 can be directly attached to the electrode surface like that disclosed in U.S. Pat. No. 5,824,473, intermediate permeation layers can be used (U.S. Pat. Nos. 5,605,662 and 5,632,957) or an electroconductive polymer can be used to coat the surface of the electrode (Caruana and Heller, J. AM. CHEM. SOC., 121; 169-174 (1999)). The probes 58 may also be attached to the electrode surface 44 through an electroconductive polymer such as poly-pyrrole as described by Livache et al., (1994) NUCLEIC ACIDS RES., 22; 2915-2921.

The oligonucleotide probes 58 may be synthesized, in situ, on the surface of the electrode 54 in either the 3′ to 5′ or 5′ to 3′ direction using the 3′-β-cyanoethyl-phosphoramidites or 5′-β-cyanoethyl-phosphoramidites and related chemistries known in the art. For array probes to be used with the arrayed primer extension reaction (see below), the probe arrays must be synthesized in the 5′ to 3′ direction in order for the 3′ terminus of the probe 58 to be available for subsequent polymerase extension. In situ synthesis of the oligonucleotides may also be performed in the 5′ to 3′ direction using nucleotide coupling chemistries that utilize 3′-photoremovable protecting groups (U.S. Pat. No. 5,908,926). Alternatively, the oligonucleotide probes may be synthesized on the standard control pore glass (CPG) in the more conventional 3′ to 5′ direction using the standard 3′-β-cyanoethyl-phosphoramidites and related chemistries (Caruthers M. et al., Method Enzymol., 154; 287-313 (1987), and U.S. Pat. Nos. 4,415,732 and 4,458,066) and incorporating a primary amine or thiol functional group onto the 5′ terminus of the oligonucleotide (Sproat et al., Nucleic Acids Res, 1987, 15, 4837, and Connolly and Rider, Nucleic Acids Res; 1985, 13, 4485). The oligonucleotides may then be covalently attached to the electrode surface via their 5′ termini using thiol or amine-dependent coupling chemistries known in the art. The density of the probes 58 on the array surface can range from about 1,000 to 200,000 probe molecules per square micron. The probe density can be controlled by adjusting the density of the reactive groups on the surface of the electrode for either the in situ synthesis post-synthesis deposition methods.

Typically, oligonucleotide probes 58 are comprised of, but not limited to, the four natural deoxyribonucleotides; deoxythymidylic acid, deoxycytidylic acid, deoxyadenylic acid and deoxyguanylic acid. The probes can also be comprised of, the ribonucleotides, uridylic acid, cytidylic acid, adenylic acid, and guanylic acid. Modified nucleosides may also be incorporated into the oligonucleotide probes. These include but are not limited to; 2′-deoxy-5-methylcytidine, 2′-deoxy-5-fluorocytidine, 2′-deoxy-5-iodocytidine, 2′-deoxy-5-fluorouridine, 2′-deoxy-5-iodo-uridine, 2′-O-methyl-5-fluorouridine, 2′-deoxy-5-iodouridine, 2′-deoxy-5(1-propynyl)uridine, 2′-O-methyl-5(1-propynyl)uridine, 2-thiothymidine, 4-thiothymidine, 2′-deoxy-5(1-propynyl)cytidine, 2′-O-methyl-5(1-propynyl)cytidine, 2′-O-methyladenosine, 2′-deoxy-2,6-diaminopurine, 2′-O-methyl-2,6-diaminopurine, 2′-deoxy-7-deazadenosine, 2′-deoxy-6methyladenosine, 2′-deoxy-8-oxoadenosine, 2′-O-methylguanosine, 2′-deoxy-7-deazaguanosine, 2′-deoxy-8-oxoguanosine, 2′-deoxyinosine or the like.

Typically, the oligonucleotide probes 58 can range in length from, but not limited to 5 to 100 nucleotides, preferably 5 to 25 nucleotides, more preferably 5 to 10 nucleotides and most preferably 5 to 8 nucleotides. Longer oligonucleotide probes are preferred for applications where the array is used in the detector or continuous flow mode (see below). Longer oligonucleotide probes are also necessary for applications where the sample contains a high sequence-complexity target mixture. Shorter oligonucleotide probes are preferred in applications where single nucleotide discrimination, such as mutation detection, is important.

The probes 58 provide an oligonucleotide array that is specific and complementary to a particular nucleic acid sequence. For example, the oligonucleotide array will contain an oligonucleotide sequence that is complementary to a specific target sequence and an individual or multiple mutation thereof.

Detection of Nucleic Acid Targets

The array of probes 58 of the present invention is intended for use in a molecular recognition-based assay for the analysis of a sample suspected of containing a target molecule or moiety such as a specific nucleic acid sequence. The probes 58 provide an oligonucleotide array for the purpose of binding and detecting a specific target nucleic acid sequence. The hybridization between the probe and target nucleic acid sequence is determined by the standard Watson-Crick hydrogen-bonding interactions.

The target nucleic acid may be genomic DNA, genomic RNA, messenger RNA, ribosomal RNA or transfer RNA, an oligonucleotide or polynucleotide of DNA or RNA generated by enzymatic process such as PCR or reverse transcription or any synthetic DNA, RNA or any combination thereof generated by chemical means. The target nucleic acid may be double stranded or single stranded. It is preferred that the target be single stranded in order to increase the efficiency of its interaction with the probe sequences. To this end, the target may contain modified nucleotides for the purposes of reducing secondary structure by disrupting intramolecular base-pairing interactions or increasing the stability of the probe-target interaction.

The architecture of the array probes may be either generic or specific with regard to the complementary target sequences that it may hybridize with. For example, an array of all possible 16,384 7-mer probe sequences could be used to interrogate targets having any sequence. The advantage of such an array is that it is not an application specific and therefore generic. Alternatively, the probe array may contain oligonucleotide sequences that are complementary to a specific target sequence or set of target sequences and individual or multiple mutations thereof. Such an array is useful in the diagnosis of specific disorders, which are characterized by the presence of a particular nucleic acid sequence. For example, the target sequence may be that of a particular exogenous disease causing agent, e.g. human immunodeficiency virus, or alternatively the target sequence may be that portion of the human genome which is known to be mutated in instances of a particular disorder, e.g. sickle cell anemia or cystic fibrosis.

In the art, the detection of nucleic acid sequences by hybridization procedures is typically carried out using oligonucleotide probes between 12 and 25 nucleotides in length. Probes of this length are generally utilized to increase the specificity of the probe-target interaction when using high sequence-complexity target mixtures and to stabilize the probe-target interaction. In the present invention, the probes can be between 4 and 100 nucleotides in length depending upon the detection mode employed. The use of shorter probes (e.g. 6-mers, 7-mers, and 8-mers) allows for a sequence-complete set of probes to be incorporated into a single array consisting of about 65,000 or fewer features, each possessing a defined sequence. For example, an array of all 6-mer sequences would consist of 4,096 (4⁶) features each possessing a unique sequence. An array of all 7-mer sequences would consist of 16,384 (4⁷) features and an array of all 8-mers would consist of 65,536 (4⁸) features. Although the use of shorter probes results in a reduced overall stability of probe-target interaction, they are better for interrogating single nucleotide changes in the target sequence. Moreover, when coupled with enzymatic processes such as a polymerase extension assay (see below), even transient probe-target interactions can be captured and recorded.

The stability of the probe-target duplex interactions can be altered by incorporating the various aforementioned modified nucleotides into the probe sequences. For example, 2,6-diaminopurine forms more stable base-pairs with thymidine than does adenosine. 2′-deoxy-5-fluorouridine, 2′-deoxy-5-bromouridine and 2′-deoxy-5-iodouridine also form more stable base-pairs with adenosine. Likewise, 2′-deoxy-5(1-propynyl)cytidine forms more stable base-pairs with guanosine. Alternatively, the stability of a base-pairing interaction can be decreased using modifications. For example, A-T base pairs can be destabilized by incorporating 2′-amino-nucleosides. Inosine can also be used in place to guanosine to destabilized G-C base pairs. Importantly, the differences in thermostablity of the probe-target duplex as a result of their sequence and base composition can be normalized using various modified nucleotides. For example, incorporating N-4-ethyl-2′-deoxycytidine has been shown to decrease the stability of G-C base pairs. Incorporating the latter can normalize the stability of any given duplex sequence to an extent where its stability is made independent of A-T and G-C content (Nguyen et al., Nucleic Acids Res. 25, 3095 (1997)). Because the probe-target interaction is bimolecular, the resulting equilibrium duplex concentration is dependent upon the initial concentration of both the probe and target species. Thus, it is likely that the probe-target duplex stability can also be controlled by adjusting the surface density of the oligonucleotide probes on the array surface.

Referring to FIGS. 1 and 3, in operation, the sample is brought into contact with the array of probes 58 by means of any system conventional in the art, such as by means of pipettes, tubing, or microfluidic pumping technology known to those skilled in the art. In the preferred mode, the probe array is placed within a sealed container fabricated from an inert material such as plastic, creating a sample chamber where the target material can contact every probe 58 on the array surface of the chip 12. The chamber volume should be kept to a minimum to reduce the amount of sample needed for the analysis. In the preferred embodiment, the chamber volume is no greater than 200 uL, more preferably no greater than 50 uL and most preferably no greater than 10 uL.

Arrayed Hybridization Detection and Continuous Monitoring: In one embodiment, each array probe 58 possesses a detector agent. A suitable detector agent is either a compound that exhibits redox activity or a chemical moiety that is one member of a bioconjugate pair. The redox complexes may have one or more functions that can be reduce or oxidized. Typically, the redox complexes contain one or more centers, namely, a center having a chemical function that accepts and transfers electrons. Some redox complexes include the transition metal oxides and mixed oxides, e.g. the oxides of W, Ni, Rh, Ir, Nb, Mo, V; the complexes of transition metals, Cd, Mg, Cu, Co, Pd, Zn, Fe, Ru, as disclosed in U.S. Pat. No. 5,591,578.

In addition to transition metal complexes, other organic electron donors and acceptors may be covalently coupled to the nucleic acid for use in the present invention. These organic molecules include, but are not limited to riboflavin, xanthene dyes, azine dyes, acridine organge, N,N′-dimethyl-2,1-diazapyrenium dichloride (DAP²⁺), methylviologen, ethidium bromide, quinones, porphyrines, carlamine blue B hydrochloride, Bindschedler's green, Brilliant crest blue, methylene blue, Nile blue A, indigo-5,5′,7,7′-tetrasulfonic acid, safranine T, iduline scarlet, neutral red and substituted derivatives of these compounds. Specific complexes known in the art for the electronic detection of DNA include: Ru(bpy)₂CO₃ and Ru^{II}(NH₃)₄py (U.S. Pat. No. 5,770,369), Ferrocene (Ihara, et al., NUCLEIC ACIDS RESEARCH, 24, 4273-4280 (1996)), and [Co(bpy)₃ ⁼³] and [Co(phen)₃ ⁼³] (Millan and Mikkelsen, ANAL. CHEM; 65, 2317-2323 (1993)).

The electrochemically active moiety can be attached to the probe 58 at any nucleotide position via the nucleotide base, ribose ring or phosphate backbone. In the preferred mode, the electrochemically active moiety is attached to either the 3′ or 5′ terminal nucleotide of the probe 58, whichever is free in solution.

The nucleic acid sample solution is applied to the surface of the array of the device 10 in a buffered solution and allowed to hybridize to the arrayed probes 58 according to methods known in the art (Lockhart et al., NATURE BIOTECHNOL, 14; 1675-1680 (1996), Cronin et al., HUMAN MUTATION, 7; 244-255 (1996)). Typical target hybridization conditions range from 1 to 100 nM nucleic acid target in a buffer containing 3 to 6×SSPE (6×SSPE contains; 0.9 M NaCl, 60 mM NaH₂PO₄, 6 mM EDTA). The hybridization buffer may also contain CTAB at concentrations ranging from 5 to 10 mM or detergents such as SDS or Triton X-100 ranging in concentration from 0.005% to 0.01%. The hybridization is carried out at 30 to 60° C. for 30 minutes to 12 hours. In the detection mode, the array is washed with a buffered solution containing 3 to 6×SSPE containing 0.005 to 0.02% SDS at 20 to 60° C. to remove the unhybridized target material. The exact hybridization conditions will depend upon the probe length and the specific application being employed. In the continuous monitoring mode, the sample is continuously passed over the array of probes 58 at a defined flow rate, e.g. 1 to 100 μL/min, while maintaining a hybridization buffer composition and hybridization temperature that ensures specific binding of the target sequences to the arrayed probes.

The change in the electronic properties of the detector agent is then monitored using the integrated circuitry of device 10. For detector agents that are redox-active such as ferrocene, the resulting changes in the redox potential of the detector agent can be monitored using cyclic voltammetry. It is known in the art that the redox potentials of oligonucleotide probes possessing ferrocene can be dependent upon the local environment of the ferocene moiety and hence sensitive to the presence or absence of a hybridized complementary strand of nucleic acid (Ihara et al., NUCLEIC ACIDS RES, 24, 4273-4280, 1996)).

Arrayed Polymerase Extension Reaction: The general arrayed primer extension (APEX) procedure is one which is known in the art, as evidenced by Shumaker, et al., HUMAN MUTATION, 7:346-354 (1996) and Pasteine, et al., GENOME RESEARCH, 7:606-614 (1997).

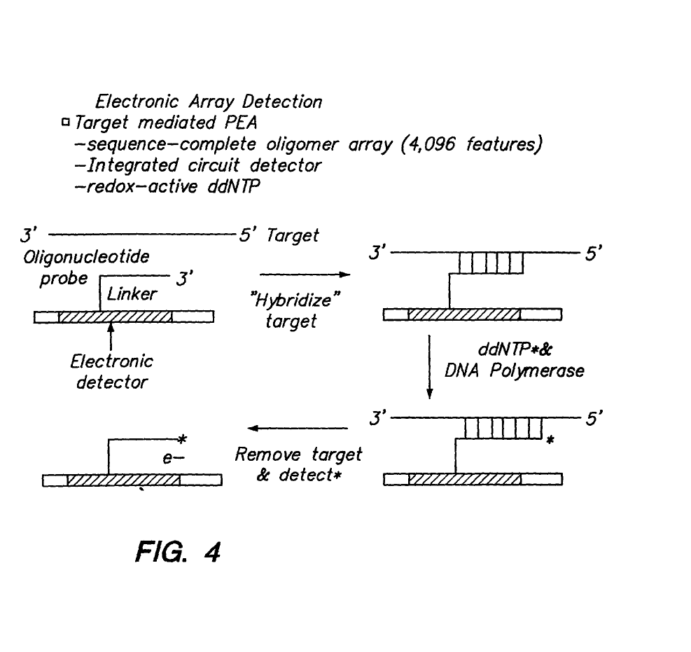

Referring to FIG. 4 where the probe 58 is a short oligonucleotide (e.g. 6-mer, 7-mer, and/or 8-mer) and the sample is a nucleic acid polymer, the sample is subjected to an APEX procedure using a suitable polymerase in buffered solution containing one or more nucleotide triphosphates having an electrochemically active moiety attached or associated thereto, which extends the arrayed probe by at least one nucleotide in a target sequence dependent manner. The electrochemically active moiety is then detected.

The present invention differs from the conventional APEX procedure in two important ways. First, in the present invention, the oligonucleotide probes are between 5 and 10 nucleotides in length, more preferably between 5 and 8 nucleotides in length and most preferably between 5 and 7 nucleotides in length. Second, the nucleotide triphosphates used to extend the probes 58 contain a detector agent, or can subsequently bind a detector agent, which is electrochemically active. Following the polymerase extension reaction, the target nucleic acid and residual nucleotide analogues are washed away from the array surface of device 10 and the electrochemical properties of each probe 58 at the electrode surface 18 are measured using the associated drive and sense circuitry within the array chip of device 10, as previously described.

The polymerase extension reaction is carried out on the surface of the array of electrodes 18 of probes 58 in a sealed container having a volume no greater than 200 uL, more preferably no greater than 50 uL and most preferably no greater than 10 uL. The buffer composition and pH will depend upon largely upon the type of polymerase employed. For example, the optimal buffer conditions for the Taq DNA polymerase are: 25 mM (pH 9.3 @ 25° C.), 50 mM KCl and 2.0 mM MgCl₂. Buffer conditions for the Bst DNA polymerase are 20 mM Tris-Cl (pH 8.8 @ 25° C.), 10 mM KCl, 10 mM (NH₄)₂SO₄, 2.0 mM MgCl₂ and 0.1% Triton X-100. The buffer conditions may also depend upon the composition of the target and probe sequences.

The nucleic acid polymerizing enzyme may be any enzyme capable of catalyzing the polymerization reaction using the four naturally-occurring nucleotide triphosphates and/or modified nucleotide triphosphates that result in chain termination. Preferably, but without limiting the scope of the present invention, the enzyme is a primer/DNA template dependent DNA polymerase, or a primer/RNA template dependent reverse transcriptase. Specific examples include E. coli DNA polymerase I, E. coli DNA polymerase I Large Fragment (Klenow fragment), Thermus aquaticus (Taq) DNA polymerase, Thermus flavus (Tfl) DNA polymerase, Thermus Thermophilus (Tth) Dna polymerase, Thermococcus litoralis (Tli) DNA polymerase, Pyrococcus furiosus (Pfu) DNA polymerase, Vent® DNA polymerase, phage T7 DNA polymerase, Bacillus stearothermophilus (Bst) DNA polymersae, AMV Reverse Transcriptase and MMLV Reverse Transciptase. The polymerase concentration may range from about 1 to 1000 nM depending upon the affinity of the polymerase for the probe-target duplex and target nucleic acid. It is preferred that the polymerase have a greater affinity for the probe-target duplex than the free target in solution. This will likely increase the overall reaction rate and efficiency of the assay.

The chain-terminating nucleotide triphosphate analogue may be a nucleotide selected from the group consisting of the four standard didexoynucleotide triphosphate analogue (ddATP, ddGTP, ddCTP, ddTTP) or any other nucleotide analogue known in the art that is efficiently incorporated by a polymerase in a target-sequence dependent manner yet prevents further extension at the 3′ terminus of the oligonucleotide. In one embodiment, the aforementioned detector agents may be directly attached to the chain-terminating nucleotide triphosphate analogue via the C5 position of pyrimidines or the C8 position of purines using chemistries known in the art.

In another embodiment, the chain-terminating nucleotide triphosphate is associated with a high affinity ligand that specifically binds a bioconjugate of the detector agent. Examples of suitable high affinity ligand-bioconjugate pairs are the biotin-streptavidin pair and the digoxigenin-antidigoxigenin pair (Kessler, Advances in Mutagenesis, Berlin/Heidelber; Springer-Verlag; 105-152 (1990)). For the biotin-streptavidin pair, the chain-terminating nucleotide would possess the high affinity binding moiety, biotin (attached via the C5 position of pyrimidines or the C8 position of purines), and the detector agent (e.g. ferrocene) would be conjugated to the streptavidin molecule. For the digoxigenin-antidigoxigenin pair, the digoxigenin hapten moiety would be attached to the chain-terminating nucleotide and the detector agent would be associated with the antidigoxigenin. Phenylboronic acid complexes may also be used for preparing other high affinity ligand-bioconjugate pairs (U.S. Pat. No. 5,594,151).

In addition to the aforementioned transition metal complexes and organic electron donors and acceptors, the bioconjugate may include enzyme-amplified systems in which the enzyme catalyzes some type of oxidation-reduction reaction. For example, reduction of hydrogen peroxide by target-bound horseradish peroxidase (HRP) is one known embodiment of this approach (de Lumley-Woodyear et al., J. Am. Chem. Soc., 118:5504-5505 (1996). In another example, the cofactors pyrroquinoline quinone and flavin adenine dinucleotide may be incorporated into the oligonucleotide probe and subsequently reconstituted with apo-glucose oxidase similar to that described by Willner et al., J. Am. Chem. Soc., 118:10321-10322, (1996). Alternatively, a bioconjugate could be formed between the apo-glucose oxidase enzyme and the streptavidin molecule. The bioconjugate would bind a biotin moiety on the chain terminating nucleotide of the probe and catalyze an oxidation-reduction reaction in the presence of the redox cofactors pyrroquinoline quinone and flavin adenine dinucleotide.

For the polymerase extension assay, the nucleotide triphosphate analogue concentration will range from 1 to 500 uM depending upon the binding constant (Kd) of the analogue for the polymerase. Lower concentrations of the analogues are preferred in order to minimize any inhibitor effect that the analogues may have on the polymerase activity and minimize the electronic background resulting from residual nucleotide analogue during the detection steps.

The polymerase extension reaction temperature will range from 4° C. to 65° C. depending upon the specific properties of the polymerase employed. It is preferred that the reactions be performed at temperatures as high as possible in order to reduce target secondary structure which can block interactions with the complementary probes. Thermophylic polymerases are preferred at these elevated temperatures since they will be more stable for longer incubation times.

The target nucleic acid concentration will range from 1×10⁻⁴ to 1×10⁻⁶ mg/mL depending upon the sequence complexity of the target and the type of assay being performed.

The reaction times can range from 5 minutes to 24 hours depending upon overall polymerase extension reaction rate that is achieved under the defined reaction conditions. For example, elevated incubation temperatures will reduce the stability of the probe-target interaction and hence overall duplex concentration. This will result in a reduced overall rate of extension and thus require longer incubation times in order to generate an amount probe extension product that can be detected by its associated electrode.

Following the polymerase extension reaction, the target, residual nucleotide triphosphate analogue and polymerase are washed away from the array surface of device 10. The wash solution will be buffered at a pH between 4 and 12 and contain sufficient mono and divalent salts to remove the reaction components from the surface of the array. It is preferred that the wash solution contain some detergent such as sodium dodecylsulfate (SDS), Triton X-100 or the like. Small amounts of organic solvents such as acetonitrile may also be added in order to disrupt any non-specific hydrophobic binding of the target and nucleotide analogues to the array surface.

Where a high affinity ligand detector agent is incorporated into the oligonucleotide probe 58, e.g. biotin, after the aforementioned washing, the resultant probe 58 with the high affinity ligand detector agent are treated with the complementary component thereto, e.g. streptavidin, having a redox moiety incorporated therewith to form probes 58 having an incorporated bio-conjugate, e.g. avidin-biotin conjugate. Residual streptavidin bio-conjugate is removed by washing the array surface at 25° C. for 30 to 120 minutes with a buffered solution containing 25 mM Tris-Cl (between pH 7-9), 150 mM NaCl, BSA ranging from 0.01 to 0.15% and Tween® from 0.01 to 0.1%.

Electrochemical Measurement

As discussed above, electrogenerated detection methods known in the art, such as voltammetry, potentiometry and amperometry can be employed with the present invention using device 10. The electrode 18 is part of an integrated circuit that is capable of addressing each site individually or in combination which controls and monitors the relevant parameters such as voltage, current or capacitance.

Cyclic voltammetry has been used to detect the presence of redox active probes such as ferrocene derivatized oligonucleotides in a hybridization assay (Ihara, et al., Nucleic Acids Res, 24; 4273-4280 (1996)). Amperometry has been used to detect redox labeled oligonucleotides (U.S. Pat. No. 5,824,473) as well as peroxidase enzyme amplified electro-reduction reactions (de Lumley-Woodyear, et al., J. Am. Chem. Soc. (1996) 118:5504-5505). Typical cyclic voltammetry is performed at a scan rate between 10 and 100 mV/s in 1 to 50 mM buffered solutions of Tris-Cl or phosphate at a pH between 6 and 9. Salts such as NaCl and MgCl₂ can also be included at concentrations ranging from 1 and 500 mM.

As mentioned above, in one embodiment a plurality of electrodes supported by a semiconductor substrate are brought into proximity with a reaction medium comprising a sample suspected of containing the target molecule. Each of the electrodes comprises at least one target probe. A plurality of cells within the semiconductor substrate are selectively addressed to apply a stimulus to each of the electrodes to activate a predetermined redox active moiety that is associated with an electrode and to detect, by means of the electrodes, corresponding responses produced as a result of the activation of the redox active moieties. The magnitude of the corresponding responses indicates the presence or absence of the target molecule in the sample. The stimulus may be voltage or current and the corresponding response may be current or voltage, respectively.

In one embodiment the cell is addressed digitally. The stimulus may be applied using an analog bus, which cooperates with circuitry on or off the semiconductor substrate to apply the stimulus to the electrode. The corresponding response is detected using an analog bus, which cooperates with circuitry on or off the semiconductor substrate to detect the corresponding response from the electrode. Alternatively, the stimulus may be applied using a digital bus, which cooperates with circuitry on or off the semiconductor substrate to apply the stimulus to the electrode. In this embodiment, the corresponding response is detected using a digital bus, which cooperates with circuitry on or off the semiconductor substrate to detect the corresponding response from the electrode, and wherein the cell includes an analog-to-digital converter. In another approach the stimulus is applied using one of an analog bus or a digital bus with a digital-to-analog converter in the cell, which cooperates with circuitry on or off the semiconductor substrate to apply the stimulus to the electrode. The corresponding response is detected using the other of an analog bus or a digital bus and an analog-to-digital converter in the cell, which cooperates with circuitry on or off the semiconductor substrate to detect the corresponding response from the electrode. In one embodiment the stimulus is stored in the cell.

Consistent with the methods described herein the redox active moiety may be incorporated into the target probe prior to bringing a plurality of electrodes supported by a semiconductor substrate into proximity with a reaction medium comprising a sample suspected of containing the target molecule. Alternatively, the redox active moiety may be incorporated into the target probe subsequent to bringing a plurality of electrodes supported by a semiconductor substrate into proximity with a reaction medium comprising a sample suspected of containing the target molecule.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. All publications and patent applications cited in this specification are herein incorporated by reference as if each individual publication or patent application were specifically and individually indicated to be incorporated by reference.

What is claimed is:

1. A method for detecting a target molecule, said method comprising:
(a) bringing a plurality of electrodes supported by a semiconductor substrate into proximity with a reaction medium comprising a sample suspected of containing said target molecule, each of said electrodes comprising at least one target probe,
(b) selectively addressing a plurality of cells within said semiconductor substrate
(i) to apply a stimulus to each of said electrodes to activate a predetermined redox active moiety that is associated with an electrode, and
(ii) to detect, by means of said electrodes, corresponding responses produced as a result of said activation of said redox active moieties, the magnitude of said corresponding responses indicating the presence or absence of said target molecule in said sample.

2. A method according to claim 1 wherein said stimulus is voltage or current and said corresponding response is current or voltage, respectively.

3. A method according to claim 1 wherein said cell is addressed digitally.

4. A method according to claim 3 wherein said stimulus is applied using an analog bus, which cooperates with circuitry on or off said semiconductor substrate to apply said stimulus to said electrode, and wherein said corresponding response is detected using an analog bus, which cooperates with circuitry on or off said semiconductor substrate to detect said corresponding response from said electrode.

5. A method according to claim 3 wherein said stimulus is applied using a digital bus, which cooperates with circuitry on or off said semiconductor substrate to apply said stimulus to said electrode, and wherein said corresponding response is detected using a digital bus, which cooperates with circuitry on or off said semiconductor substrate to detect said corresponding response from said electrode, and wherein said cell includes an analog-to-digital converter.

6. A method according to claim 3 wherein said stimulus is applied using one of an analog bus or a digital bus with a digital-to-analog converter in said cell, which cooperates with circuitry on or off said semiconductor substrate to apply said stimulus to said electrode, and wherein said corresponding response is detected using the other of an analog bus or a digital bus and an analog-to-digital converter in said cell, which cooperates with circuitry on or off said semiconductor substrate to detect said corresponding response from said electrode.

7. A method according to claim 3 wherein said stimulus is stored in said cell.

8. A method according to claim 1 wherein said redox active moiety is incorporated into said target probe prior to step (a).

9. A method according to claim 1 wherein said redox active moiety is incorporated into said target probe subsequent to step (a).

10. A method according to claim 4 wherein target probe comprises an oligonucleotide and said redox active moiety or precursor thereto is incorporated into said target probe by means of a target dependent polymerase extension reaction.

11. A method according to claim 1 wherein said detecting comprises the use of voltammetry or potentiometry.

12. A method for detecting a target molecule, said method comprising:
(a) bringing a plurality of electrodes supported by a semiconductor substrate into proximity with a reaction medium comprising a sample suspected of containing said target molecule, each of said electrodes comprising at least one target probe,
(b) selectively applying electrical signals to each of said electrodes to activate a predetermined redox active moiety that is associated with an electrode, and
(c) detecting, by means of said electrodes, corresponding electrical signals produced as a result of said activation of said redox active moieties, the magnitude of said corresponding electrical signals indicating the presence or absence of said target molecule in said sample.

13. A method according to claim 12 wherein said selectively applied electrical signals are voltages and said corresponding electrical signals are current or potential difference or a combination thereof.

14. A method according to claim 12 wherein said redox active moiety is incorporated into said target probe prior to step (a).

15. A method according to claim 12 wherein said redox active moiety is incorporated into said target probe subsequent to step (a).

16. A method according to claim 15 wherein target probe comprises an oligonucleotide and said redox active moiety or precursor thereto is incorporated into said target probe by means of a target dependent polymerase extension reaction.

17. A method according to claim 12 wherein said each electrical signals are selectively applied to said electrodes by means of a plurality of digital analog converters, each of which is selectively electrically coupled to said electrodes.

18. A method according to claim 12 wherein said electrical signals are selectively applied to said electrodes by one or more analog converters which is selectively electrically coupled to said electrodes.

19. A method according to claim 12 further comprising (i) sending an address to address decoders on said semiconductor substrate, said address decoders being in communication with each of said cells, (ii) sending an item of numerical data to storage means in each of a plurality of cells within said semiconductor substrate by means of a data bus, said item of numerical data participating in the selection of a voltage to be applied to said electrodes, and (iii) sending an address to address decoders on said semiconductor substrate, said address decoders being in communication with said storage means, whereby said item of numerical data is stored in said storage means and electrical signals are selectively applied to each of said electrodes to activate a predetermined redox active moiety that is associated with an electrode.

20. A method according to claim 19 wherein an analog bus is employed and said item of numerical data identifies said analog bus that connects to said electrode.

21. A method according to claim 12 wherein said detecting comprises the use of voltammetry or potentiometry.

22. A method for detecting a target nucleic acid, said method comprising:
(a) bringing a plurality of electrodes supported by a semiconductor substrate into proximity with a reaction medium comprising a sample suspected of containing said target molecule, each of said electrodes comprising at least one oligonucleotide probe,
(b) sending an item of numerical data to each of a plurality of cells within said semiconductor substrate by means of a data bus, said item of numerical data participating in the selection of a voltage to be applied to said electrodes,
(c) sending an address to address decoders on said semiconductor substrate, said address decoders being in communication with each of said cells, whereby electrical signals are selectively applied to each of said electrodes to activate a predetermined redox active moiety that is associated with an electrode, and
(d) detecting, by means of said electrodes, corresponding electrical signals produced as a result of said activation of said redox active moieties, the magnitude of said corresponding electrical signals indicating the presence or absence of said target nucleic acid in said sample.

23. A method according to claim 22 wherein said selectively applied electric signals are voltages and said corresponding electric signals are current or potential difference or a combination thereof.

24. A method according to claim 22 wherein said redox active moiety is incorporated into said oligonucleotide probe prior to step (a).

25. A method according to claim 22 wherein said redox active moiety is incorporated into said oligonucleotide probe subsequent to step (a).

26. A method according to claim 25 wherein said redox active moiety or precursor thereto is incorporated into said oligonucleotide probe by means of a target dependent polymerase extension reaction.

27. A method according to claim 22 wherein said electrical signal is selectively applied to said electrode by means of a digital analog converter which is electrically coupled to said electrode and is associated with said cell.

28. A method according to claim 22 wherein said electrical signal is selectively applied to said electrode by an analog converter which is electrically coupled to said electrode and is associated with said cell.

29. A method according to claim 22 wherein said item of numerical data is representative of an electrical signal.

30. A method according to claim 22 wherein an analog bus is employed and said item of numerical data identifies said analog bus that connects to said electrode.

31. A method according to claim 22 wherein said detecting comprises the use of voltammetry or potentiometry.

32. A method according to claim 22 wherein said at least one oligonucleotide binds a defined target nucleic acid sequence.

33. A method according to claim 22 wherein said at least one oligonucleotide binds an undefined target nucleic acid sequence.

34. A method according to claim 22 wherein said at least one oligonucleotide is part of a set of sequence specific oligonucleotides of predetermined length.

35. A method for detecting a target nucleic acid, said method comprising:
(a) bringing a plurality of electrodes supported by a semiconductor substrate into proximity with a reaction medium comprising a sample suspected of containing said target molecule, each of said electrodes comprising at least one oligonucleotide probe wherein each of said oligonucleotide probes comprises a redox active moiety,
(b) sending an item of numerical data to storage means in each of a plurality of cells within said semiconductor substrate by means of a data bus, said item of numerical data participating in the selection of a voltage to be applied to said electrodes,
(c) sending an address to address decoders on said semiconductor substrate, said address decoders being in communication with said storage means, whereby said item of numerical data is stored in said storage means and voltages are selectively applied to each of said electrodes to activate said redox active moieties, and
(d) detecting, by means of said electrodes, corresponding current or difference in potential or a combination thereof produced as a result of said activation of said redox active moieties, the magnitude of said current or potential difference indicating the presence or absence of said target nucleic acid in said sample.

36. A method according to claim 35 wherein said voltages are selectively applied to said electrodes by means of a plurality of digital analog converters, each of which is electrically coupled to an electrode and is associated with a cell.

37. A method according to claim 35 wherein said voltages are selectively applied to said electrodes by an analog converter which is electrically coupled to said electrode and is associated with said cell.

38. A method according to claim 35 wherein said item of numerical data is representative of a voltage.

39. A method according to claim 35 wherein an analog bus is employed and said item of numerical data identifies which analog bus connects to which electrode.

40. A method according to claim 35 wherein said detecting comprises the use of voltammetry or potentiometry.

41. A method according to claim 35 wherein said at least one oligonucleotide binds a defined target nucleic acid sequence.

42. A method according to claim 35 wherein said at least one oligonucleotide binds an undefined target nucleic acid sequence.

43. A method according to claim 35 wherein said at least one oligonucleotide is part of a set of sequence specific oligonucleotides of predetermined length.

44. A method for detecting a target nucleic acid, said method comprising:
(a) bringing a plurality of electrodes supported by a semiconductor substrate into proximity with a reaction medium comprising a sample suspected of containing said target molecule, each of said electrodes comprising at least one oligonucleotide probe,
(b) conducting a polymerase extension reaction to incorporate in each of said oligonucleotide probes, to which a target nucleic acid is bound, a redox active moiety or precursor thereof,
(c) sending an item of numerical data to storage means in each of a plurality of cells within said semiconductor substrate by means of a data bus, said item of numerical data participating in the selection of a voltage to be applied to said electrodes,
(d) sending an address to address decoders on said semiconductor substrate, said address decoders being in communication with said storage means, whereby said item of numerical data is stored in said storage means and voltages are selectively applied to each of said electrodes to activate redox active moieties associated with said electrodes, and
(e) detecting, by means of said electrodes, corresponding current or difference in potential or a combination thereof produced as a result of said activation of said redox active moieties, the magnitude of said current or potential difference indicating the presence or absence of said target nucleic acid in said sample.

45. A method according to claim 44 wherein prior to step (c) said electrodes are washed.

46. A method according to claim 44 wherein said polymerase extension reaction comprises having present in said medium nucleotide triphosphate analogs comprising said redox active moiety or precursor thereto.

47. A method according to claim 46 wherein said medium is incubated at temperature wherein said target nucleic acid hybridizes to respective oligonucleotide probes, which are extended by the addition of a respective nucleotide triphosphate analog.

48. A method according to claim 44 wherein said precursor comprises a small organic molecule and said method comprises having present in said medium a binding partner for said small organic molecule, said binding partner comprising a redox active moiety.

49. A method according to claim 44 wherein said precursor comprises a binding partner for a small organic molecule and said method comprises having present in said medium a small organic molecule comprising a redox active moiety.

50. A method according to claim 44 wherein said at least one oligonucleotide binds a defined target nucleic acid sequence.

51. A method according to claim 44 wherein said at least one oligonucleotide binds an undefined target nucleic acid sequence.

52. A method according to claim 44 wherein said at least one oligonucleotide is part of a set of sequence specific oligonucleotides of predetermined length.

53. A method for identifying target nucleic acids in a sample, which comprises:
(a) applying said sample to a plurality of test sites, each of said test sites comprising an oligonucleotide probe attached to an electrode, each of said electrodes being part of a surface of an integrated circuit, each of said oligonucleotide probes being capable of specifically binding to a target nucleic acid molecular structure, such that each of said test sites has oligonucleotide probes which specifically bind to a different target molecular structure;
(b) incubating said sample on said test sites in the presence of a polymerase and nucleotide triphosphate analogs comprising a redox active moiety or a precursor thereof to extend each oligonucleotide probe, to which a target nucleic acid molecular structure is bound, and to associate said redox active moiety or precursor thereof with each extended oligonucleotide probe, to which a target nucleic acid molecular structure is bound, with the proviso that, when said nucleotide triphosphate analogs comprise a precursor, a binding partner for said precursor is added wherein said binding partner comprises a redox active moiety;
(c) applying a voltage to each of said test sites by means of circuitry associated with said integrated circuit, said voltage being sufficient to activate said redox active moiety associated with an electrode; and
(d) detecting by means of said integrated circuit a current or difference in potential or a combination thereof at each of said test sites, the magnitude of which is related to the presence of said target nucleic acids in said sample.

54. A method according to claim 53 wherein said oligonucleotide probes are DNA probes or RNA probes.

55. A method according to claim 53 wherein said detecting comprises the use of voltammetry or potentiometry.

56. A method according to claim 53 wherein said integrated circuit comprises:
(a′) a semiconductor substrate supporting a plurality of electrodes and
(b′) a plurality of cells within said semiconductor substrate, wherein said integrated circuit comprises upon a single substrate:
(i) a plurality of digital analog converters, each electrically coupled to a respective electrode and each being associated with a respective cell,
(ii) address decoders in communication with each of said cells,
(iii) a data bus for delivering binary numerical data to each of said cells,
(iv) address buses for delivering addresses to said address decoders, and
(v) storage means in each of said cells for storing said numerical data, said storage means being in communication with said digital analog converter in said cell; and wherein steps (c) and (d) comprise:
(a″) sending binary numerical data to said storage means of each said cells by means of said data bus, said binary numerical data being representative of an electrical signal,
(b″) sending addresses to said address decoders whereby said binary numerical data is stored in said storage means and electric signals are selectively applied to each of said electrodes by means of said digital analog converters to activate said redox active moiety associated with an electrode and
(c″) detecting, by means of said electrodes, corresponding electrical signals produced as a result of said activation of said redox active moieties, the magnitude of said corresponding electrical signals indicating the presence or absence of said target molecule in said sample.

57. A method of testing a sample for the presence of target nucleic acids, said method comprising:
(a) applying said sample to an array of test sites in multiple locations on a surface of an integrated circuit, each site having oligonucleotide probes formed therein of known binding characteristics wherein the oligonucleotide probes in each test site differ from the oligonucleotide probes in other test sites in a known predetermined manner such that the test site location of oligonucleotide probes and their binding characteristics are known;
(b) treating each test site to which a target nucleic acid is bound, to extend the length of each oligonucleotide probe thereby incorporating an electronically responsive detector agent into each of said oligonucleotides;
(c) applying an electrical signal to each of said test sites by means of circuitry associated with said integrated circuit, said electrical signal being sufficient to activate said electronically responsive detector agent associated with an electrode; and
(d) detecting by means of said integrated circuit a change in electronic properties of the test sites resulting from the binding of target nucleic acid to lengthened oligonucleotide probes in the test sites by detection circuitry coupled to individual test sites to determine which target nucleic acid has bound to a test site; whereby the presence of a multiplicity of different target nucleic acids in the sample is detected.

58. A method according to claim 57 wherein said oligonucleotide probes are DNA probes or RNA probes.

59. A method according to claim 57 wherein said detecting comprises the use of voltammetry or potentiometry.

60. A device comprising:
(a) a semiconductor substrate,
(b) at least one surface having associated therewith a redox active moiety,
(c) an electrode adjacent said surface and supported by said semiconductor substrate,
(d) a cell within said semiconductor substrate,
(e) a digital analog converter to which said electrode is electrically coupled, said digital analog converter being associated with said cell,
(f) an address decoder in communication with said cell,
(g) a data bus for delivering an item of numerical data to said cell,
(h) an address bus for delivering an address to said address decoder, and
(i) means for monitoring said surface.

61. A device according to claim 60 wherein said means for monitoring comprises an analog bus.
